(19) United States
(12) Patent Application Publication
(10) Pub. No.: US 2009/0125071 A1
(43) Pub. Date: May 14, 2009

(54) SHAPE-CHANGING ANATOMICAL ANCHOR

(76) Inventors: David M. Skinlo, North Logan, UT (US); Roger Pisamwongs, Valencia, CA (US); Thomas Weisel, Ventura, CA (US)

Correspondence Address: KOPPEL, PATRICK, HEYBL & DAWSON, 2815 Townsgate Road, SUITE 215, Westlake Village, CA, 91361-5827, US

(21) Appl. No.: 12/256,270

(22) Filed: Oct. 22, 2008

Related U.S. Application Data
(60) Provisional application No. 61/000,248, filed on Oct. 23, 2007.

(51) Int. Cl. A61B 17/04; A61B 17/08

(52) U.S. Cl. 606/300; 606/301; 606/151; 606/325; 606/74

(57) ABSTRACT

A shape-changing anatomical anchor includes an activation means arranged to convert the anchor from a de-activated state to an activated state, and one or more members which extend away from the activation means and thereby change the shape of the anchor when the anchor is activated. The anchor is installed within bone and/or soft tissue when de-activated; when activated, the shape change acts to increase the force with which the anchor is retained within the tissue in which it is installed. Both piloted and non-piloted versions are described.

### RELATED APPLICATIONS

This application claims the benefit of provisional patent application No. 61/000,248 to D. Skinlo et al., filed Oct. 23, 2007.

### BACKGROUND OF THE INVENTION

1. Field of the Invention

This invention relates generally to fixation devices which are implanted within the body.

2. Description of the Related Art

Conventionally, fixation between bone and bone, or between bone and soft tissue (such as muscle or tendon) when used in interference-type applications or approximation is created using screw-type implants. These screws generally require pilot holes and a driver to install and provide the required fixation or interference.

A conventional interference screw implant 10 for orthopedic fixation applications is shown in FIG. 1. The screw has deep cut threads 12 which allow it to hold in soft tissue to bone and in some cases bone to bone. The screw is driven via a feature which allows a high torque to be applied to the screw, such as a hex socket 14 located on the top of the screw body 16. These screws are generally constructed of stainless steel, titanium or possibly bioresorbable materials, with the materials selected for biocompatibility and long term mechanical strength and fixation. This type of screw is generally manufactured using conventional machining, which includes lathes, mills and possibly injection molding of non-metallic materials.

Though this type of implant has proved very useful, it has several drawbacks. For example, the use of pilot drills and holes, while effective in improving implant retention, adds additional steps and expense to the surgical procedure.

Another significant drawback of current implant designs relates to their use with soft tissue, in that soft tissue may be damaged by the screw threads as the screw moves along the tissue during installation. This is a problem especially for interference-type screw applications.

One additional drawback common to the majority of existing implant types is that they only partially take advantage of the natural bone structure to improve retention. As mentioned previously, most implants are installed into pilot holes which have been pre-drilled to a specific size. These holes are drilled into bone which is composed of basically two types: cortical and cancellous. These two types of bone vary greatly in their mechanical properties, and traditional implants fail to capitalize on those variations. Cortical bone is a dense bone material and forms a type of shell which protects the much softer cancellous bone. Traditional implants typically create an opening in the cortical bone which is relatively large relative to the implant, which tends to reduce the influence of the cortical bone on overall implant retention strength.

There have been many advances in the design of these sorts of implants, and significant research has been conducted regarding the design of traditional screw-type implants. This research is primarily focused on addressing the above issues to help support the overall goal of longevity and overall implant retention over time. Several advances have been made which address some of the concerns summarized above, such as tapered thread designs, rounded threads, and various drive mechanisms. However, all of these changes are iterations on a traditional screw-design theme, and as such do not fully overcome the above-noted drawbacks.

### SUMMARY OF THE INVENTION

The present invention is directed to a shape-changing anatomical implant useful for the fixation of bone and soft tissue, which overcomes or mitigates some of the drawbacks noted above.

The present implant, referred to herein as an ‘anchor’, has activated and de-activated states. The anchor includes an activation means which converts the anchor from its de-activated state to its activated state, and one or more members which extend away from the activation means and thereby change the shape of the anchor when the anchor is activated. The anchor is suitable for installation within bone and/or soft tissue when in its de-activated state, and then when activated, the shape change acts to increase the force with which the anchor is retained within the bone and/or soft tissue in which it is installed.

Several different embodiments are described, including some which include a pointed tip with which the anchor can be driven into tissue, and others which require a pilot hole. The embodiments employ several different types of activation means, as well as several different member-types. However, all embodiments are arranged to be suitable for installation into a particular tissue when de-activated, and to be firmly anchored within the tissue when installed and activated.

These and other features, aspects, and advantages of the present invention will become better understood with reference to the following description and claims.

### BRIEF DESCRIPTION OF THE DRAWINGS

FIG. 1 is an illustration of a conventional interference screw.

FIGS. 2a-2c are perspective, plan and detailed views, respectively, of a shape-changing anchor per the present invention which employs wedge-shaped body portions.

FIGS. 3a-3b are elevation and plan views, respectively, of another possible embodiment of a shape-changing anchor per the present invention which employs wedge-shaped body portions.

FIGS. 3c and 3d are plan views illustrating the operation of one possible embodiment of a spring mechanism which temporarily allows the anchor to return to the deactivated state, as might be used with an anchor as shown in FIGS. 2a-2c or 3a-3b.

FIGS. 4a-4d are elevation views illustrating the use of a mating tool with a shape-changing anchor per the present invention which employs wedge-shaped body portions.

FIGS. 5a-5d are perspective, side elevation and front elevation views of a non-piloted version of a shape-changing anchor per the present invention.

FIGS. 6a-6g are perspective views of a piloted version of a shape-changing anchor per the present invention and its various components.

FIGS. 6i-6j are plan views of the anchor of FIGS. 6a-6g, illustrating the anchor's camming action.

FIGS. 6k and 6L are plan and corresponding sectional views of an embodiment of a shape-changing anchor per the present invention which employs leaf springs as an activation means.

FIGS. 7a and 7b are plan views of another possible embodiment of a shape-changing anchor per the present invention, shown in its de-activated and activated states, respectively.

FIG. 8 is a plan view of another possible embodiment of a shape-changing anchor per the present invention.

FIGS. 9a-9c are plan, sectional and magnified views, respectively, of another possible embodiment of a shape-changing anchor per the present invention.

FIGS. 10a-10c are perspective, schematic and plan views, respectively, of a shape-changing anchor per the present invention which employs spike-shaped members.

FIGS. 11a-11c are perspective, plan and sectional views, respectively, of another possible embodiment of a shape-changing anchor per the present invention which employs spike-shaped members.

FIGS. 12a and 12b are plan and sectional views, respectively, of one possible embodiment of a nut which inhibits the de-activation of a shape-changing anchor per the present invention.

FIGS. 13a and 13b are plan and sectional views, respectively, of another possible embodiment of a nut which inhibits the de-activation of a shape-changing anchor per the present invention.

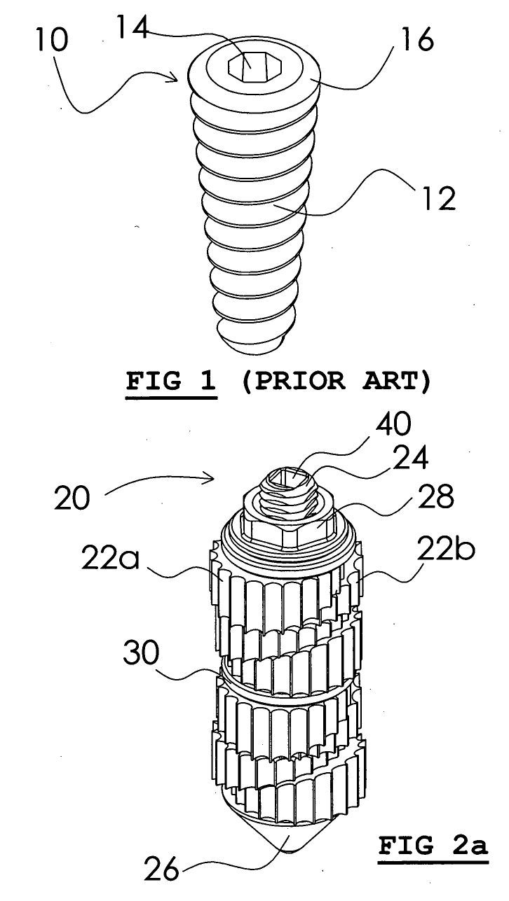

### DETAILED DESCRIPTION OF THE INVENTION

The present shape-changing anatomical anchor is useful for the fixation of bone and soft tissue. Several exemplary embodiments are described and many others are possible; however, common to all embodiments is that each has ‘activated’ and ‘de-activated’ states, and is equipped with a means by which the anchor can be converted from its de-activated to its activated state. The anchor also has one or more members which are arranged to extend away from the activation means and thereby change the shape of the anchor when the anchor is converted to its activated state.

An anchor as described herein is suitable for installation within bone and/or soft tissue when in its de-activated state. Then, once installed and activated, the anchor's shape change acts to increase the force with which the anchor is retained within the bone and/or soft tissue, thereby making it more difficult for the anchor to be pulled out or dislocated. In some embodiments, the activation means is arranged such that it can also convert the anchor from its activated state back to its de-activated state. This can be useful if there is a need to remove or relocate the anchor after it has been installed and activated.

Embodiments are described which are to be installed directly within bone and/or soft tissue without the use of a pilot hole, while others are arranged such that at least a portion of the anchor is installed in a pilot hole formed within the tissue in which the anchor is to be installed.

The present anchor is useful for many different fixation applications. For example, an anchor as described herein could be used to enable the orthopedic fixation of soft tissue to bone, the fixation of bone to bone, or the fixation of bone to tissue which has been inserted in a bone tunnel formed in the bone. For example, the anchor can be used in an ACL/PCL replacement procedure, where it acts to fix the tendon graft bundle into the femoral or tibial canal. Another possible application would be to use the anchor in soft tissue to suspend a bladder neck, as a means of treating incontinence.

Anchors as described herein can be made from a number of different materials. Examples of materials that might be used include metals, plastics, PEEK, bioresorbables, and bioconductives.

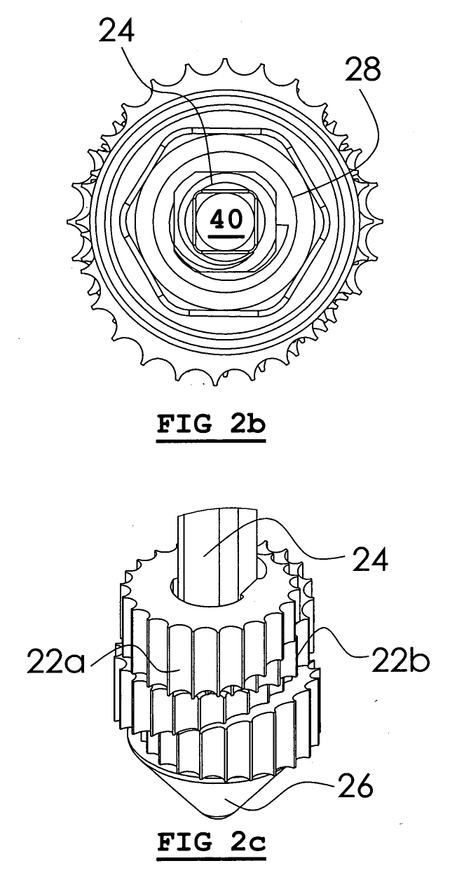

One possible embodiment 20 is shown in FIG. 2a-2c. In this implementation, the anchor's members comprise at least one pair of wedge-shaped body portions 22a, 22b; four such pairs are shown in FIG. 2a, though more or fewer pairs may be used as needed for a given application.

Each wedge-shaped body portion has at least one sloped surface, with a sloped surface of one body portion of each pair stacked atop a sloped surface of the other body portion of each pair, such that the pair of wedge-shaped body portions tends to slide along their sloped surfaces in opposite directions when subjected to a force applied substantially perpendicular to the directions of movement. Thus, as oriented in FIG. 2a, body portions 22a and 22b slide to the left and right, respectively, in response to a force applied vertically.

The activation means includes a central shaft 24 that runs through each of the wedge-shaped body portions. The activation force is then applied along an axis parallel to that of the central shaft. Here, central shaft 24 is threaded at one end and includes a bottom portion 26 at its other end, and the wedge-shaped body portions are disposed around the shaft between the bottom portion and a nut 28 threaded onto the top of the shaft. The activation force is then applied by threading nut 28 towards bottom portion 26 so as to compress the wedge-shaped body portions against each other, causing them to slide radially outwards, away from shaft 24; a plan view of the anchor with its wedge-shaped body portions extended away from shaft 24 is shown in FIG. 2b. This changes the shape of the anchor, and acts to increase the force with which the anchor is retained within the bone and/or soft tissue in which it was installed. A pilot hole would typically be required for the installation of an anchor of this type.

In general, an anchor of this type is arranged such that, when de-activated, the force applied substantially perpendicular to the directions of movement is less than that required to force the wedge-shaped body portions away from the central shaft. But, when activated, the applied force is sufficient to force the wedge-shaped body portions to expand radially away from the central shaft.

The central shaft 24 and the wedge-shaped body portions are preferably arranged such that the body portions cannot rotate about the shaft; this is illustrated in FIG. 2c. This arrangement allows the wedge-shaped body portions to slide laterally, but does not allow them to rotate around the shaft. This anti-rotation feature forces the wedge-shaped body portions to extend away from the shaft in known directions, thereby ensuring that the anchor has full radial expansion. An anchor of this type preferably has at least two pairs of wedge-shaped body portions, arranged such that, when activated, at least four of the body portions extend away from the shaft in four different directions. This arrangement ensures an almost complete radial expansion, thereby tending to ensure proper bone or tissue contact and a strong retention force.

As shown in FIG. 2a, the anchor can include one or more intermediate planar surface portions 30 affixed to shaft 24 between nut 28 and bottom portion 26. At least one pair of wedge-shaped body portions are then placed on the shaft between the nut and intermediate planar surface portion 30, and at least one pair of body portions is placed between surface portion 30 and bottom portion 26.

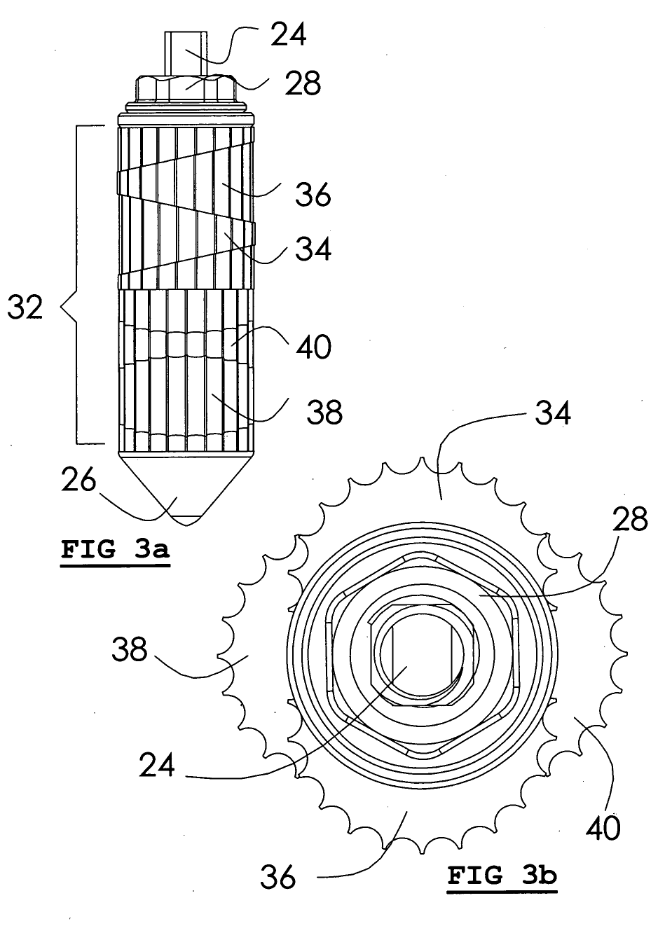

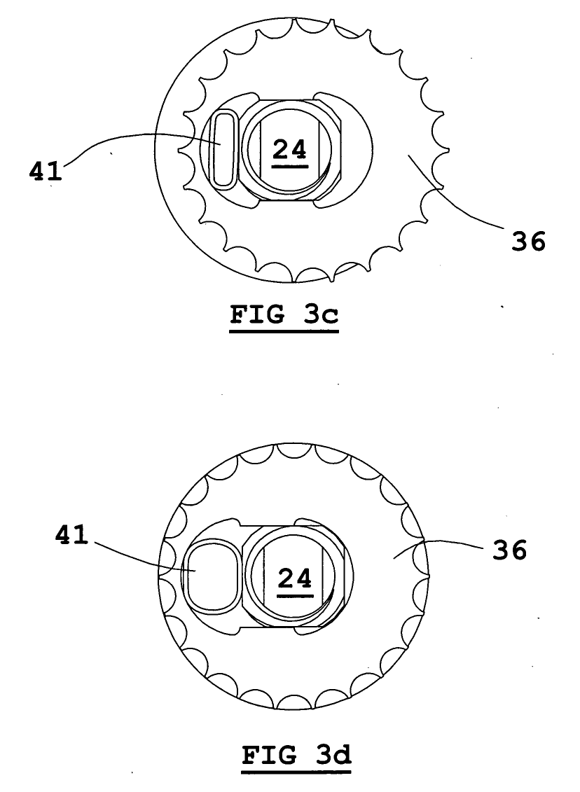

Alternatively, as shown in FIG. 3a, wedge-shaped body portions 32 can be stacked between nut 28 and bottom portion 26 with no intermediate planar surface portions. FIG. 3a depicts the anchor in its de-activated state, with none of its wedge-shaped body portions extended away from central shaft 24. In the plan view of FIG. 3b, nut 28 has been threaded towards bottom portion 26, applying sufficient force to the stack such that the body portions are forced to extend radially away from the shaft. In this example, body portions 34, 36, 38 and 40 are forced forward, backward, left and right, respectively, with respect to the central shaft.

It may be desirable to be able to return an activated anchor back to its de-activated state, to adjust the location of the anchor, for example, or to remove it. One possible means by which this process can be assisted is illustrated in FIGS. 3c and 3d; only one wedge-shaped body portion 36 is shown for clarity. In the plan view of FIG. 3c, a compressible feature 41, located in a gap between shaft 24 and the inner diameter of wedge 36, is in a compressed state when wedge 36 is in its activated position. To return the anchor to its deactivated position, the means by which vertical force is applied to the wedge stack is loosened, and the lateral force applied by compressed feature 41 acts to nudge wedge 36 back to its de-activated state, as shown in FIG. 3d. Feature 41 can be inherently compressible, such as a deformable plastic tube (Teflon, etc.), a spring made out of stainless steel, or a shape-memory material such as Nitinol, or may be arranged such that its transition between compressed and uncompressed states is user-controlled.

The activation means of an anchor per the present invention preferably includes a torque feature arranged to receive a mating tool which, when engaged with the torque feature and operated, acts to activate the anchor. Such a torque feature is seen in FIGS. 2a and 2b, as a square socket 40 recessed into the top of central shaft 24. A square-shafted tool arranged to fit socket 40 can be provided which, when engaged, enables central shaft 24 to be more easily rotated.

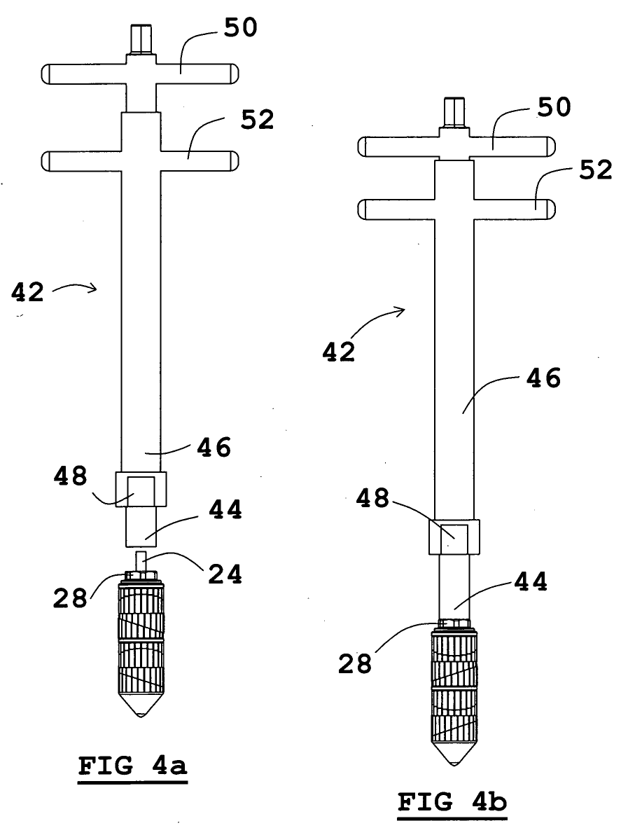

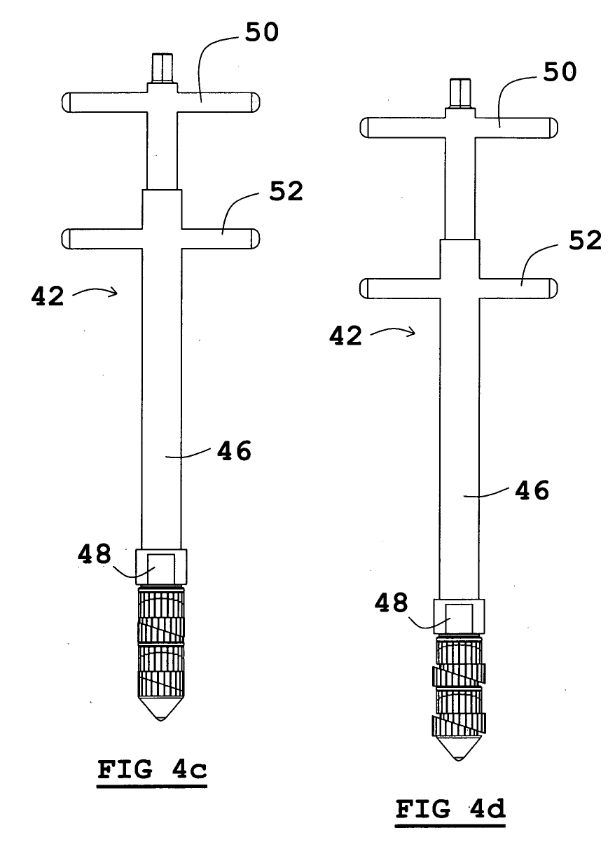

This is illustrated in more detail in FIGS. 4a-4d. Here, a tool 42 includes concentric shafts 44 and 46, with shaft 46 able to slide up and down over shaft 44. Shaft 44 is arranged to engage a torque feature such as square socket 40 at the top of the anchor's central shaft 24, while shaft 46 includes a socket portion 48 arranged to fit over the perimeter of the nut 28 threaded onto the top of shaft 46. Each shaft preferably includes a handle 50, 52 with which the shaft can be rotated.

In FIG. 4b, tool 42 is shown with shaft 44 engaged with the anchor's torque feature, and in FIG. 4c, shaft 46 has been positioned so that socket portion 48 is in place over nut 28. To activate the anchor, handle 52 is rotated while handle 50 is held so that it does not rotate, such that nut 28 is threaded down the shaft, thereby forcing the wedge-shaped body portions to extend radially away from shaft 24, as shown in FIG. 4d. Note that the tool and torque feature arrangement of FIGS. 4a-4d is merely exemplary; there are many other arrangements with which an anchor per the present invention could be activated and deactivated.

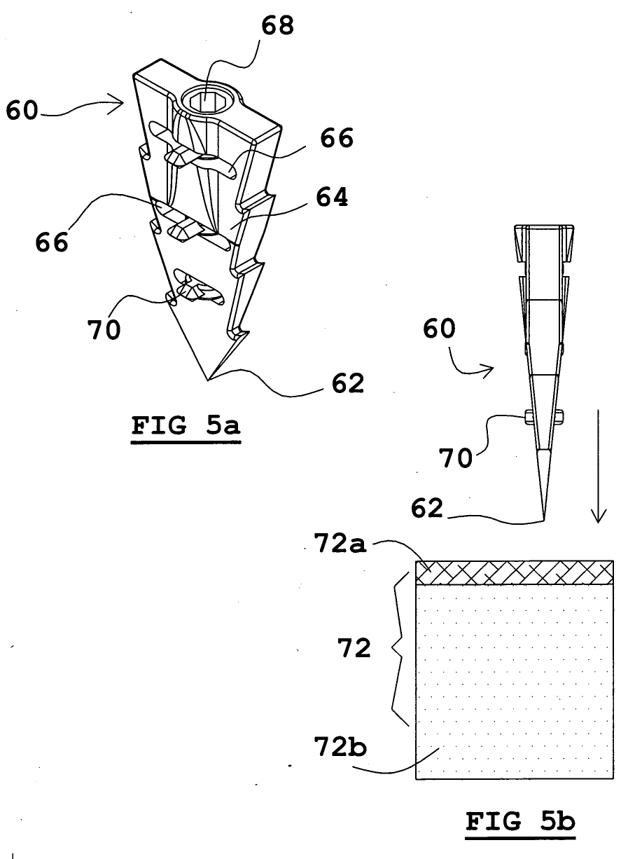

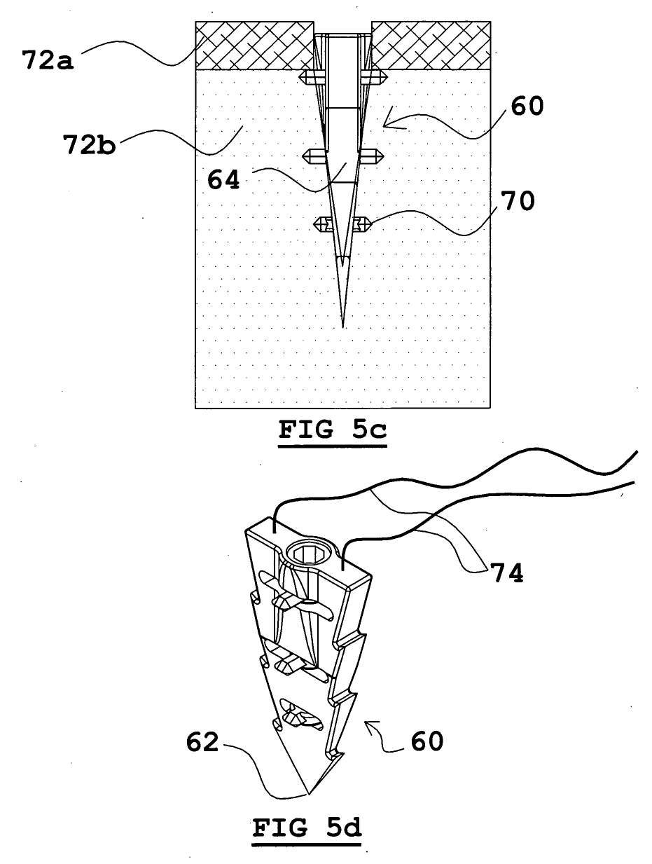

An anchor per the present invention may be a ‘piloted’ type—i.e., arranged to be installed within a pilot hole, or a ‘non-piloted type’—which is installed directly within bone and/or soft tissue without the use of a pilot hole. An example of the latter type is shown in FIGS. 5a-5d. Here, the anchor 60 includes a pointed tip 62, which enables the anchor to be driven into the bone and/or soft tissue in which it is to be installed. For this exemplary embodiment, the anchor's body 64 includes one or more slots or recesses 66, and its activation means comprising a rotatable shaft 68 to which the anchor's members 70 are coupled. The anchor is arranged such that, when de-activated, the shaft is in a first position such that the members are largely flush with body 64 and contained within respective recesses 66, and when activated, the shaft is rotated away from the first position such that the members extend away from body 64. The anchor is shown in its activated state in FIG. 5a.

The anchor 60 is shown in its de-activated state in FIG. 5b. The pointed tip 62 allows the anchor to be driven into tissue such as bone 72 (which includes high strength cortical bone 72a and softer cancellous bone 72b), via a mallet or slide hammer, for example. Once in position, the anchor would be activated by rotating shaft 68, preferably with a tool which mates with a torque feature such as the hex socket seen at the top of shaft 68 in FIG. 5a. As shown in FIG. 5c, rotating shaft 68 causes small blade-type members 70 to be extended away from body 64, thereby changing the shape of the anchor and increasing the force with which the anchor is retained within the bone and/or soft tissue in which it was installed.

The present anchor, as well as the other anchor embodiments described below, may be arranged such that its members can be locked in their extended positions or inhibited from returning to their de-activated positions once the anchor has been activated; such a locking or inhibiting means may be permanent, or temporary—with the possibility of being overridden by the user. For example, a set of mating flats or detents or similar features could be employed to keep the activation means from returning to its de-activated position once the anchor has been activated.

One possible application for this type of anchor would be for tissue approximation to bone, in which case sutures 74 could be attached to the anchor as shown in FIG. 5d.

The anchor's shape-changing design improves anchor retention in two ways: (1) by increasing the surface area/contact area with the softer cancellous bone 72b, and (2) by allowing the device to bear up on the high strength cortical bone 72a (as shown in FIG. 5c). This improved mechanical retention is one of the primary advantages of the present shape-changing anchor, whether in piloted or non-piloted form.

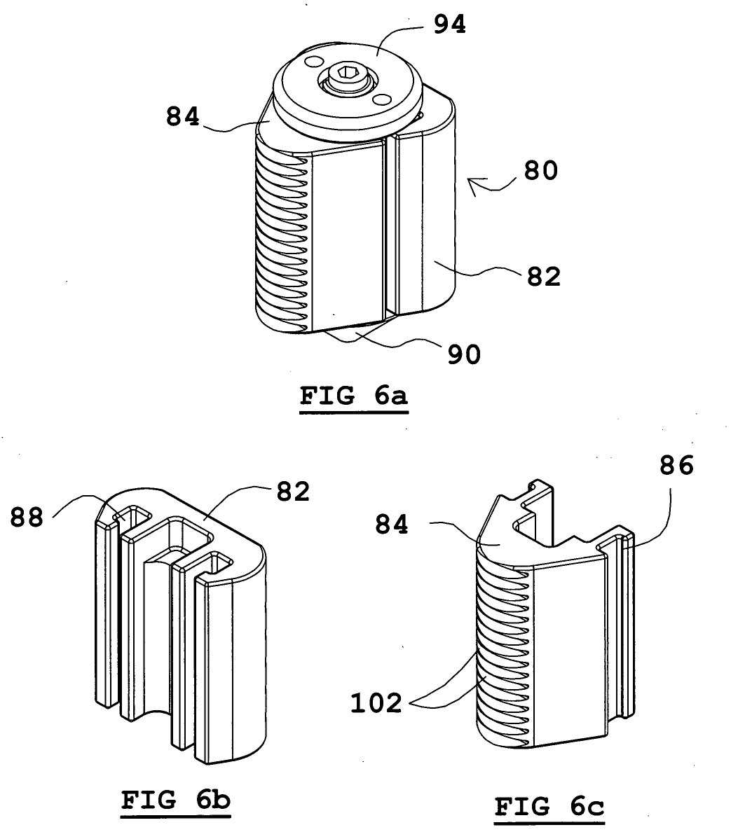

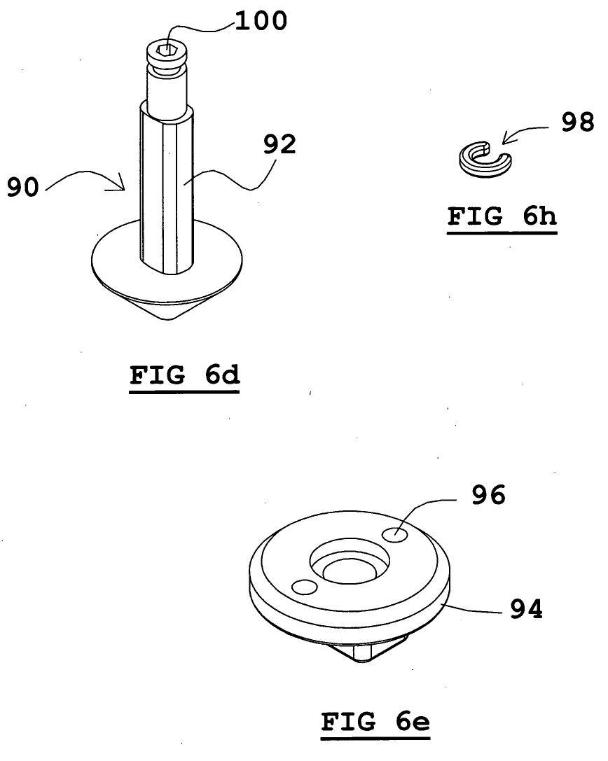

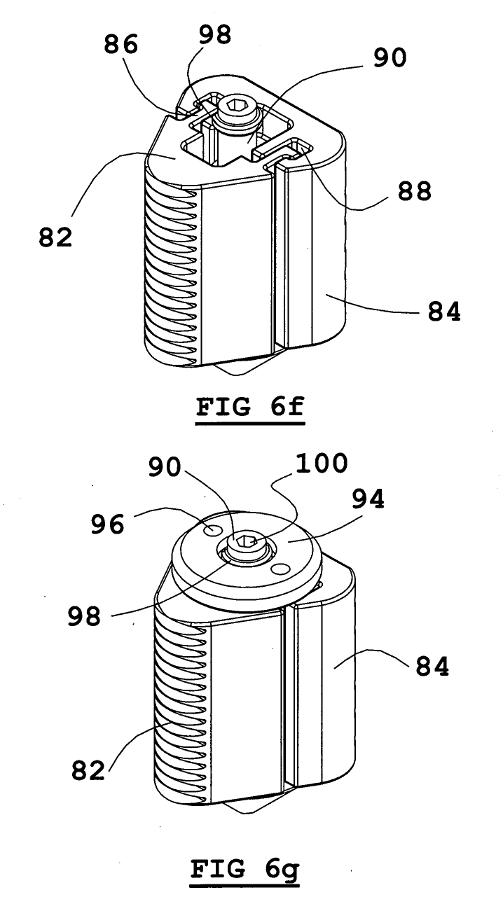

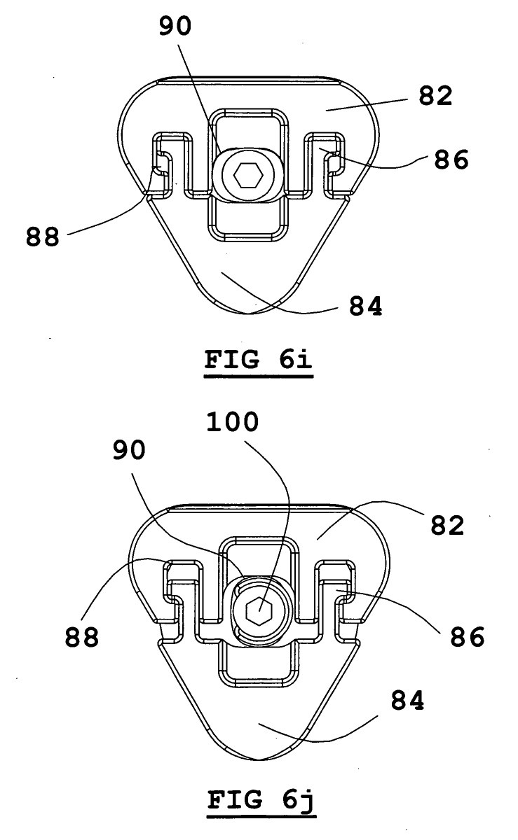

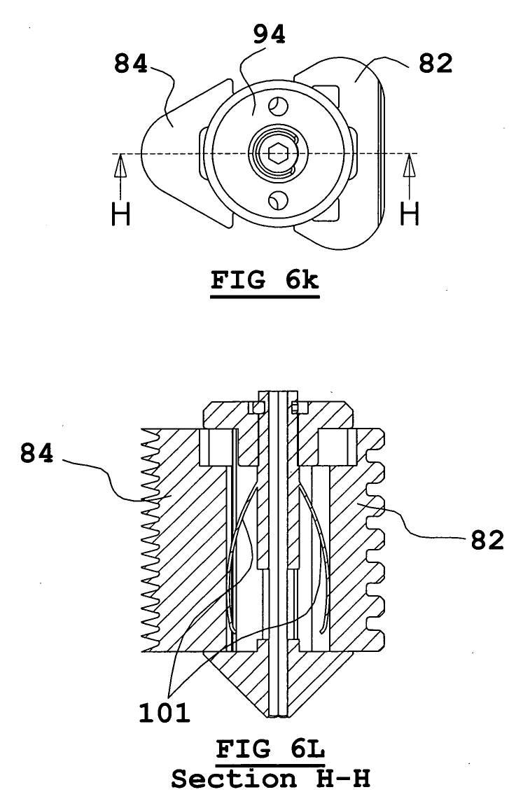

Another possible piloted embodiment 80 is shown in FIGS. 6a-6j. This anchor's body design has a blunt tip, which is primarily due to the desire to provide an anchor with a very high surface area and having very high tissue retention, without causing tissue damage during or after anchor installation. The basic principles remain the same: insert anchor into pilot hole, and activate the anchor to generate tissue retention forces.

An assembled anchor is shown in FIG. 6a. The anchor's members comprise at least two body portions: a back portion 82 (shown in detail in FIG. 6b) and a front portion 84 (shown in detail in FIG. 6c), which are arranged to be nested and interlocked such that the distance each body portion can travel radially away from the anchor's activation means when the anchor is activated is limited by the other body portions. In this example, key features 86 on body portion 84 are arranged to fit within slot features 88 on body portion 82 when the anchor is assembled.

There are numerous methods by which this anchor can be activated. In this example, the desired shape-changing effect is obtained by means of a central camshaft around which body portions 82 and 84 are disposed. The anchor is arranged such that, when de-activated, the shaft is in a first position such that the body portions are not extended away from the camshaft, and when activated, the camshaft is rotated such that the body portions are forced away from the camshaft.

An exemplary camshaft 90 is shown in FIG. 6d, which provides the primary axis and camming surfaces 92 for this design. A top cap 94 (FIG. 6e) provides an upper control surface as well as containing features 96 for controlling counter torque.

By way of assembly (FIG. 6f), body portions 82 and 84 are interlocked and disposed around camshaft 90. The top cap 94 is installed over camshaft 90 (FIG. 6g), and a split ring 98 (FIG. 6h) is inserted, locking the system together. The cam shaft is preferably activated via a torque feature such as hex socket 100, with the counter torque provided by top cap 94 and features 96.

FIGS. 6i and 6j describe the cam action that defines this design. The camshaft 90 nests between the two body portions 82, 84 while in its de-activated state (FIG. 6i). During activation, camshaft 90 is rotated, preferably via a torque feature such as the hex socket 100 and the counter-rotation features 96 (not shown). Rotating camshaft 90 forces body portions 82 and 84 to separate (FIG. 6j) and thereby create the retention forces required for the anchor.

One possible alternative to a cam arrangement is shown in the plan and sectional views of FIGS. 6k and 6L, respectively. Here, the camshaft is replaced with leaf springs 101 made from a shape-changing material capable of being transformed from a first, pre-formed shape to a second, expanded shape when the anchor is activated; Nitinol is one such material. When de-activated, the leaf springs would be in their first, pre-formed shape such that the body portions are in their de-activated positions. Then, when activated, via heat from the patient's body or some external source, for example, the leaf springs transform to their second, expanded shape such that the body portions are forced to extend outward.

The anchor is preferably arranged such that at least one of its body portions includes an uneven face portion—such as serrated edges 102 shown on body portion 84 in FIG. 6c—which serves to engage the bone and/or soft tissue and tends to further increase the anchor's retention force when it is installed within the tissue and activated.

As noted above, an anchor as shown in FIG. 6a may be arranged such that the shaft and/or the body portions are locked in their extended positions or inhibited from returning to their de-activated positions once the anchor has been activated. For example, here, a set of mating flats or detents or similar features could be employed to keep camshaft 90 from returning to its first position once the anchor has been activated.

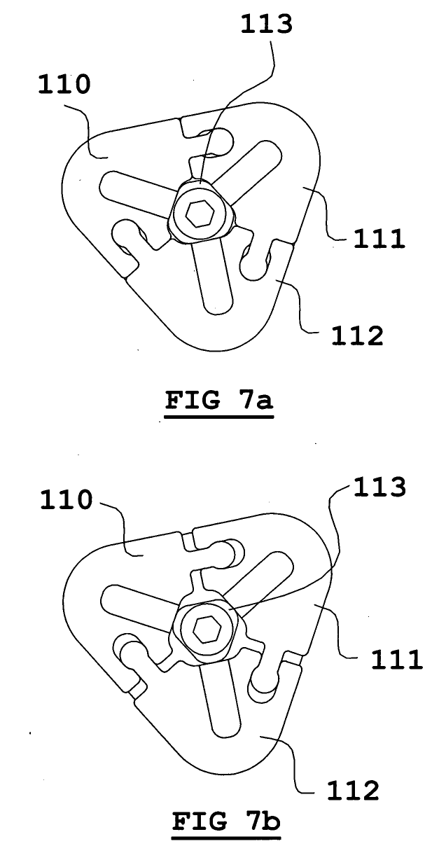

FIGS. 7a and 7b illustrate a variation on this design, and show how the number of body portions could increase to allow for different form factors for alternative anchor shapes. These include a tri-lobed design as shown in FIGS. 7a and 7b, though quad-lobed or other potential options are possible. In FIG. 7a, the anchor is in its de-activated state, with the three lobes 110, 111, 112 nested together to consume the smallest possible volume. The anchor is converted to its activated state in FIG. 7b, by rotating camshaft 113 such that lobes 110, 111, 112 are forced away from the camshaft.

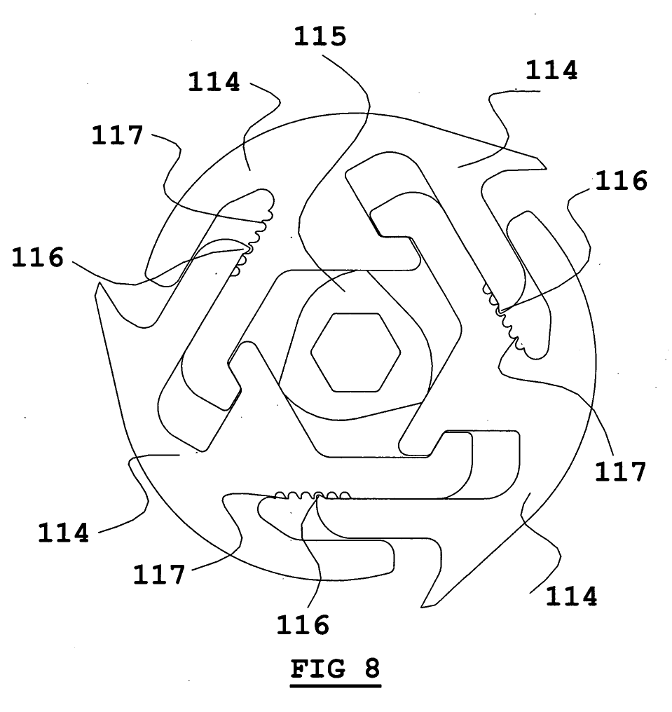

FIG. 8 illustrates another possible variation. Here, the anchor has three interlinked lobes 114, which are forced away from a central camshaft 115 when activated. Here, the shape of each of the camshaft surfaces which force body portions 114 away from the camshaft have a variable ramp, which improves mechanical advantage.

This embodiment also includes an arrangement in which body portions 14 are locked in their extended positions or inhibited from returning to their de-activated positions once the anchor has been activated. Here, each body portion 114 includes a pin 116 and teeth 117, with the anchor arranged such that the pin of one body portion engages the teeth of another body portion to form ratchet arrangements which inhibit the body portions from returning to their de-activated positions after the anchor has been activated.

Note that, instead of a camshaft, a shape-changing anchor per the present invention might utilize a screw thread having a diameter that varies along its length, to improve the mechanical advantage of the camming action.

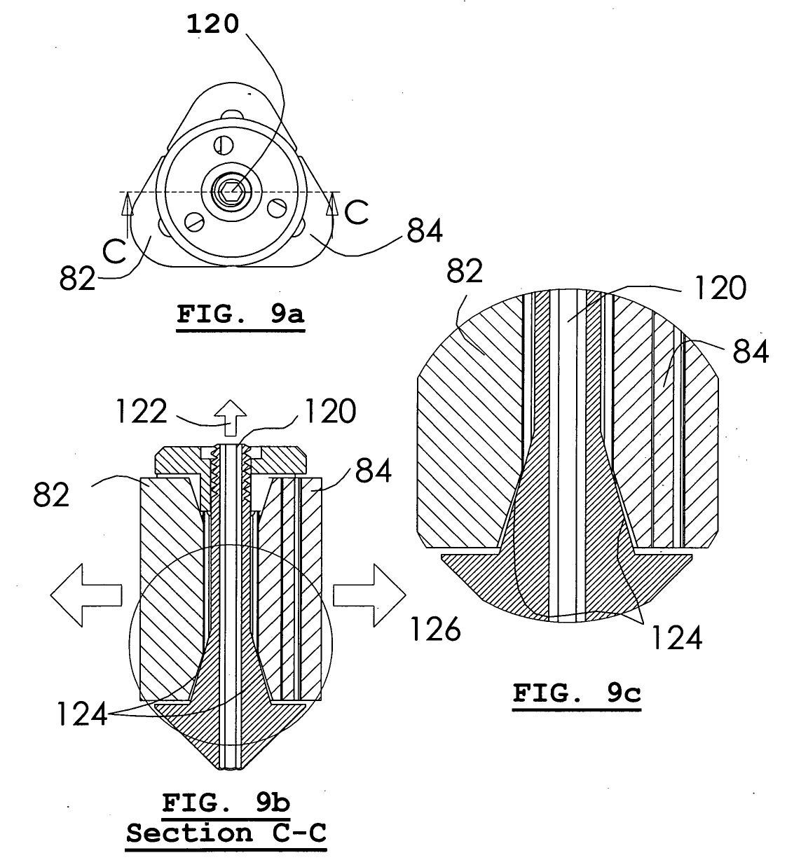

There are numerous methods by which an anchor having the general design of that shown in FIG. 6a can be activated; another possibility is illustrated in FIGS. 9a-9c; FIG. 9a is a plan view of the anchor, FIG. 9b is a sectional view cut along section line A-A in FIG. 9a, and FIG. 9c is a magnified view of a portion of FIG. 9b. In this example, the desired shape-changing effect is obtained through a ‘push/pull’ method of activation. The anchor includes a central shaft 120 around which the body portions (82, 84) are disposed. The anchor is arranged such, when de-activated, the shaft is in a first position such that body portions 82, 84 are not extended away from shaft 120, and when activated, the shaft is moved vertically along its longitudinal axis 122 such that the body portions are forced away from the central shaft.

As illustrated in FIGS. 9b-9c, body portions 82 and 84 include respective ramp portions 124, and shaft 120 has corresponding recessed areas. When the anchor is de-activated (as shown in FIGS. 9b and 9c), ramp portions 124 fit within respective recessed areas such that body portions 82, 84 are not forced away from central shaft 120. However, when activated by moving shaft 120 vertically along its longitudinal axis 122, ramp portions 124 are no longer aligned with the recessed areas; this results in shaft 120 exerting force on the ramp portions, causing body portions 82, 84 to move radially away 126 from central shaft 120.

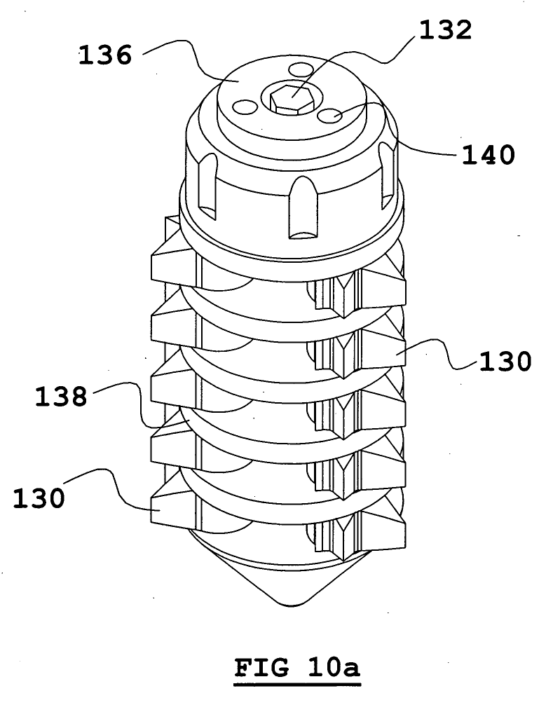

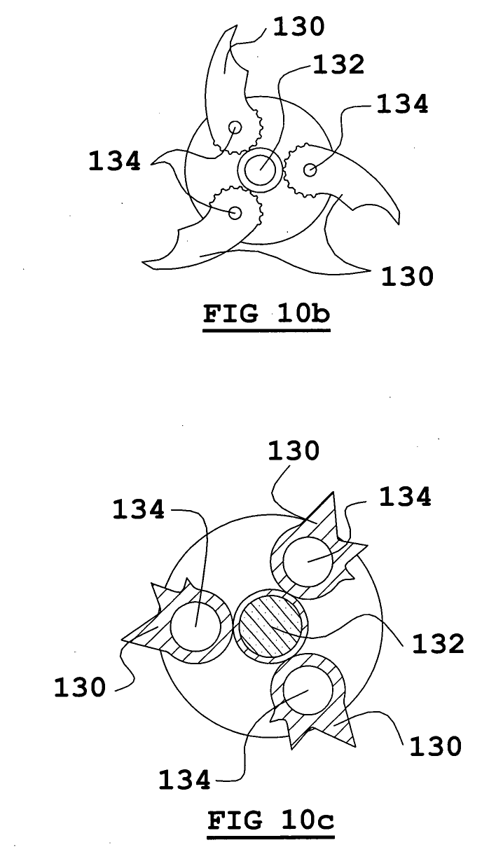

Another possible embodiment is shown in FIGS. 10a-10c: a perspective view of the overall anchor is shown in FIG. 10a, a simple schematic view of the anchor's members and activation means is shown in FIG. 10b, and a view which illustrates the interaction between the members and activation means is shown in FIG. 10c. This piloted-type anchor includes a series of spikes 130 which are engaged when the anchor is installed and activated. The anchor's activation means comprises a central drive shaft 132, and each spike is mechanically coupled to the drive shaft and arranged to pivot about a pivot point 134 and extend away from the shaft when activated. The anchor is arranged such that, when de-activated, drive shaft 132 is in a first position such that the spikes 130 are folded inward and thus not extended away from the shaft. When activated, the drive shaft is rotated such that the spikes pivot about their pivot points and extend away from the shaft, thereby changing the shape of the anchor. The extended spikes engage the tissue in which the anchor is installed, and thereby increase the force with which the anchor is retained within the tissue.

An anchor of this sort includes a top cap 136 and at least one planar surface 138 on which at least one of the pivot points and spikes resides. Planar surfaces 138 are substantially parallel to top cap 136 and preferably positioned at respective fixed distances below the cap, and central drive shaft 132 passes through each of the planar surfaces.

Some form of mechanical coupling is required between drive shaft 132 and spikes 130. For example, shaft 132 can include one or more gears, and each of spikes 130 can include a gear which meshes with a respective one of the drive shaft gears to effect the mechanical coupling.

Drive shaft 132 preferably includes a torque feature such as the hex head at the top of the shaft shown in FIG. 10a. A mating tool is preferably designed such that, when engaged with the torque feature and operated, it acts to rotate the shaft and thereby activate the anchor. The anchor preferably includes a counter-rotation feature which, when held stationary while the mating tool is operated, prevents spikes 130 from rotating around shaft 132 when the shaft is rotated. For example, the holes 140 shown in top cap 136 in FIG. 9a can serve as a counter-rotation feature.

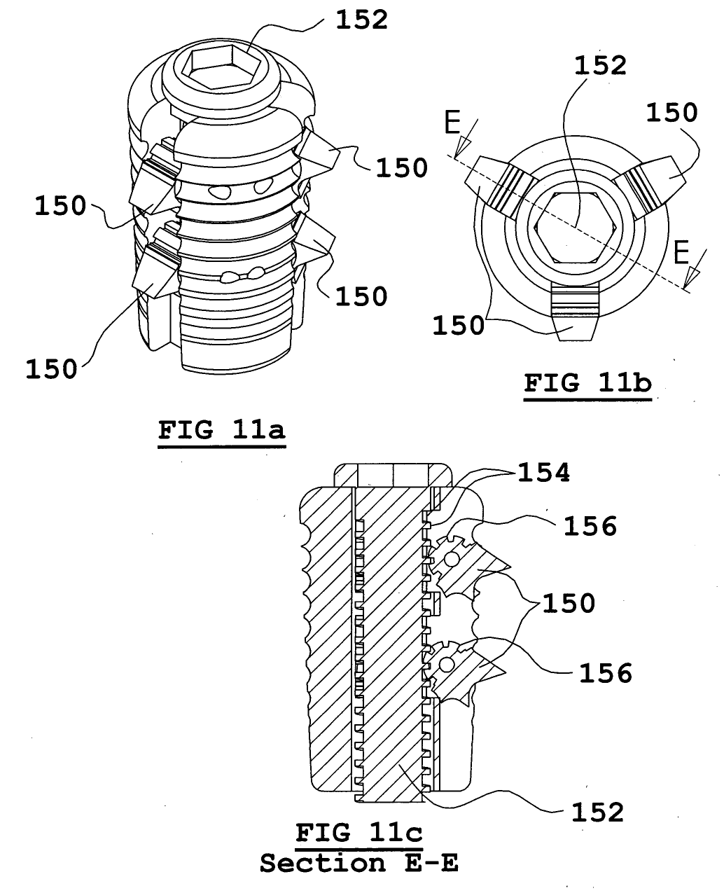

An alternative version of the ‘spike’ embodiment shown in FIGS. 10a-10c could be arranged such that, rather than pivot away from shaft 132 horizontally, the spikes could be made to deploy vertically; this is illustrated in the perspective, plan and sectional views of FIGS. 11a, 11b and 11c, respectively. That is, when de-activated, spikes 150 would be folded up against and be essentially parallel to central shaft 152. Then when activated, the spikes would unfold up or down by about 90°, such that they extend away from shaft 152. In this case, activation could be effected by providing a spiral thread 154 that engages substantially perpendicular gear portions 156 on spikes 150, so that the spikes are driven up or down when shaft 152 is rotated. Alternatively, the shaft and gear portions could be arranged such that the spikes are driven up or down by pushing or pulling the shaft vertically.

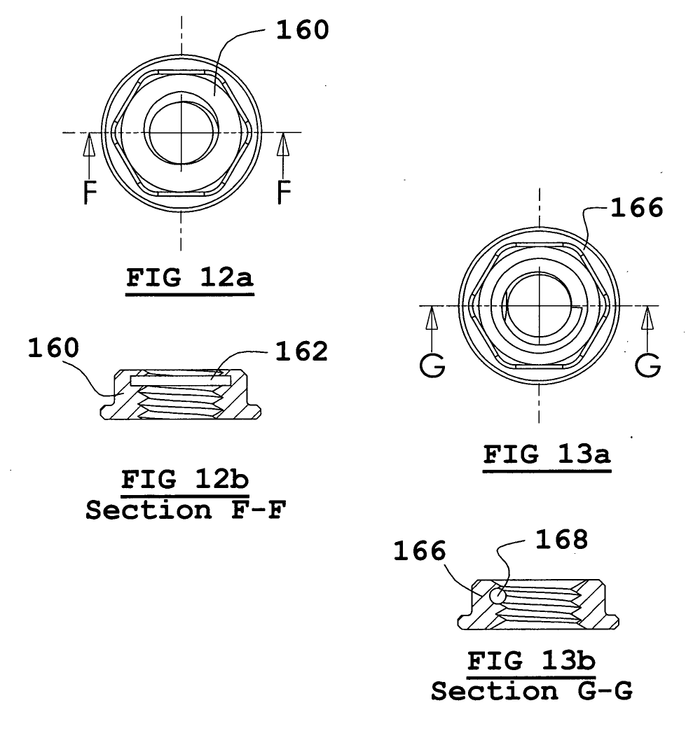

As noted above, the present anchor may be arranged such that its members can be locked in their extended positions or inhibited from returning to their de-activated positions once the anchor has been activated. There are many ways in which this can be achieved; two exemplary possibilities are shown in the plan views of FIGS. 12a and 13a, along with their corresponding sectional views 12b and 13b, respectively. In FIGS. 12a and 12b, a nut 160 includes a deformable material 162 disposed around its inner diameter. Material 162 can be, for example, formed into a ring affixed around the nut's inner diameter; a Nylok nut is one example. Deformable material 162 serves to resist the rotation of nut 160, thereby inhibiting the anchor from returning to its de-activated state. In FIGS. 13a and 13b, a nut 166 includes a notch along a portion of its inner diameter in which a deformable material 168 is placed. Material 168 can be, for example, formed into a cylindrical rod which fits into a corresponding notch and serves to interfere with the rotation of nut 166 and thereby inhibit the anchor from returning to its de-activated state. The deformable material 162, 168 can be, for example, nylon, Teflon or PEEK.

Note that the rotation inhibiting means shown in FIGS. 12, 12b, 13a and 13b are merely exemplary; many other possible embodiments are possible.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

We claim:

1. A shape-changing anatomical anchor useful for the fixation of bone and soft tissue, said anchor having activated and de-activated states, comprising:
an activation means arranged to convert said anchor from its de-activated state to its activated state; and
one or more members which are arranged to extend away from said activation means and thereby change the shape of said anchor when said anchor is converted to its activated state;
such that said anchor is suitable for installation within bone and/or soft tissue when in said de-activated state, said shape change when activated tending to increase the force with which said anchor is retained within said tissue.

2. The anchor of claim 1, wherein said activation means is further arranged to convert said anchor from its activated state to its de-activated state.

3. The anchor of claim 1, wherein at least a portion of said anchor is arranged to be installed in a pilot hole formed within the tissue in which said anchor is to be installed.

4. The anchor of claim 1, wherein said anchor is arranged to enable the fixation of soft tissue to bone.

5. The anchor of claim 1, wherein said anchor is arranged to enable the fixation of bone to bone.

6. The anchor of claim 1, wherein said anchor is arranged to enable the fixation of bone to tissue which has been inserted in a bone tunnel formed in said bone.

7. The anchor of claim 6, wherein said tissue is a tendon graft and said bone tunnel is the femoral or tibial canal.

8. The anchor of claim 1, wherein the materials from which said anchor is made are selected from the group consisting of metals, plastics, PEEK, bioresorbables, and bioconductives.

9. The anchor of claim 1, wherein said activation means includes a torque feature arranged to receive a mating tool which, when engaged with said torque feature and operated, acts to activate said anchor.

10. The anchor of claim 1, wherein said members comprise at least one pair of wedge-shaped body portions, each of said portions having at least one sloped surface, a sloped surface of one body portion of each pair stacked atop a sloped surface of the other body portion of each pair such that said pair of wedge-shaped body portions tends to slide along said sloped surfaces in opposite directions when subjected to a force applied substantially perpendicular to said directions of movement.

11. The anchor of claim 10, wherein said activation means comprises:
a central shaft that runs through each of said wedge-shaped body portions; and
a means of applying force to said wedge-shaped body portions along an axis parallel to that of said central shaft;
said anchor arranged such that, when de-activated, said applied force is less than that required to force said wedge-shaped body portions away from said central shaft, and when activated, said applied force is sufficient to force said wedge-shaped body portions to expand radially away from said central shaft.

12. The anchor of claim 1, wherein said anchor is arranged to be installed directly within said bone and/or soft tissue without the use of a pilot hole.

13. The anchor of claim 12, wherein said anchor includes a pointed tip with which said anchor can be driven into said bone and/or soft tissue.

14. The anchor of claim 13, further comprising a body which includes said pointed tip and one or more slots or recesses, said activation means comprising a rotatable shaft to which said members are coupled, said anchor arranged such that, when de-activated, said shaft is in a first position such that said members are largely flush with said body and contained within respective slots or recesses, and when activated, said shaft is rotated such that said members extend away from said body.

15. The anchor of claim 1, wherein said members comprise at least two body portions, said body portions arranged to be nested and interlocked such that the distance each body portion can travel radially away from said activation means when said anchor is activated is limited by the other body portions.

16. The anchor of claim 15, wherein said activation means comprises a central camshaft around which said body portions are disposed, said anchor arranged such that, when de-activated, said shaft is in a first position such that said body portions are not extended away from said camshaft, and when activated, said camshaft is rotated such that said body portions are forced away from said camshaft.

17. The anchor of claim 15, wherein said activation means comprises a central shaft around which said body portions are disposed, said anchor arranged such, when de-activated, said shaft is in a first position such that said body portions are not extended away from said central shaft, and when activated, said shaft is moved vertically along its longitudinal axis such that said body portions are forced away from said central shaft.

18. The anchor of claim 1, wherein said activation means comprises a central drive shaft and said members comprise at least one spike, each of which is mechanically coupled to said drive shaft and arranged to pivot about a pivot point and extend away from said shaft when activated;
such that, when de-activated, said drive shaft is in a first position such that said spikes are not extended away from said central drive shaft, and when activated, said drive shaft is rotated such that said spikes pivot about their pivot points and thereby extend away from said shaft.

19. The anchor of claim 1, wherein at least a portion of said anchor is in contact with and applies a load on cortical bone.

20. The anchor of claim 1, further comprising a securing means affixed to said anchor which enables the fixation of a particular tissue with respect to said anchor.

21. The anchor of claim 20, wherein said securing means are sutures.

22. The anchor of claim 1, wherein said anchor is arranged such that it can be locked in said activated state after said anchor has been activated.

23. The anchor of claim 1, further comprising a compressible feature positioned and arranged so as to assist in the conversion of said anchor from its activated state to its de-activated state.

24. A shape-changing anatomical anchor useful for the fixation of bone and soft tissue, said anchor having activated and de-activated states, comprising:
at least one pair of wedge-shaped body portions, each of said portions having at least one sloped surface, a sloped surface of one body portion of each pair stacked atop a sloped surface of the other body portion of each pair such that said pair of wedge-shaped body portions tends to slide along said sloped surfaces in opposite directions when subjected to a force applied substantially perpendicular to said directions of movement;
a central shaft that runs through each of said wedge-shaped body portions; and
a means of applying force to said wedge-shaped body portions along an axis parallel to that of said central shaft;
said anchor arranged such that, when de-activated, said applied force is less than that required to force said wedge-shaped body portions away from said central shaft, and when activated, said applied force is sufficient to force said wedge-shaped body portions to expand radially away from said central shaft;
said anchor suitable for installation within bone and/or soft tissue when in said de-activated state, said shape change when activated tending to increase the force with which said anchor is retained within said tissue.

25. The anchor of claim 24, wherein said central shaft and said wedge-shaped body portions are arranged such that said wedge-shaped body portions cannot rotate about said shaft.

26. The anchor of claim 25, wherein said at least one pair of wedge-shaped body portions comprises at least two pairs, said shaft and said pairs arranged such that, when activated, at least four of said wedge-shaped body portions extend away from said shaft in four different directions.

27. The anchor of claim 24, wherein said central shaft is threaded at one end and includes a bottom portion at its other end, said pairs of wedge-shaped body portions disposed around said threaded shaft between said bottom portion and a nut threaded onto said shaft;
said force applied by threading said nut towards said bottom portion so as to compress said wedge-shaped body portions against each other.

28. The anchor of claim 27, further comprising at least one additional intermediate planar surface portion affixed to said shaft between said nut and said bottom portion, at least one pair of wedge-shaped body portions disposed around said shaft between said nut and said intermediate planar surface portion, and at least one pair of wedge-shaped body portions disposed around said shaft between said intermediate planar surface portion and said bottom portion.

29. A shape-changing anatomical anchor useful for the fixation of bone and soft tissue, said anchor having activated and de-activated states, comprising:
a body having one or more slots or recesses and a pointed tip with which said anchor can be driven into said bone and/or soft tissue;
a rotatable shaft coupled to said body; and
one or more members which are coupled to said rotatable shaft;
said anchor arranged such that, when de-activated, said shaft is in a first position such that said members are largely flush with said body and contained within respective slots or recesses, and when activated, said shaft is rotated such that said members extend away from said body and thereby change the shape of said anchor;
said anchor suitable for installation within said bone and/or soft tissue when in said de-activated state, said shape change when activated tending to increase the force with which said anchor is retained within said tissue.

30. The anchor of claim 29, wherein said shaft includes a torque feature arranged to receive a mating tool which, when engaged with said torque feature and operated, acts to rotate said shaft.

31. The anchor of claim 29, wherein said members comprise respective blades.

32. The anchor of claim 29, wherein said anchor is arranged such that said members are locked in their extended positions or inhibited from returning to their de-activated positions after said anchor has been activated.

33. A shape-changing anatomical anchor useful for the fixation of bone and soft tissue, said anchor having activated and de-activated states, comprising:
at least two body portions;
a central shaft, said body portions disposed around said central shaft;
said anchor arranged such that, when de-activated, said shaft is in a first position such that said body portions are not extended away from said central shaft, and when activated, said shaft is moved such that said body portions are forced away from said shaft and thereby change the shape of said anchor;
said body portions arranged to be nested and interlocked such that the distance each body portion can travel radially away from said central shaft when said anchor is activated is limited by the other body portions;
said anchor suitable for installation within said bone and/or soft tissue when in said de-activated state, said shape change when activated tending to increase the force with which said anchor is retained within said bone and/or soft tissue.

34. The anchor of claim 33, wherein said body portions include respective ramp portions and said central shaft includes recessed areas, said anchor arranged such that, when de-activated and said shaft is in said first position, said ramp portions fit within respective recessed areas such that said body portions are not extended away from said central shaft, and when activated, said shaft is moved vertically along its longitudinal axis such that said ramp portions no longer fit within said recessed areas, thereby forcing said body portions away from said central shaft.

35. The anchor of claim 33, wherein said central shaft is a camshaft, said anchor arranged such that, when de-activated, said shaft is in a first position such that said body portions are not extended away from said central shaft, and when activated, said camshaft is rotated such that said body portions are forced away from said camshaft.

36. The anchor of claim 35, wherein said camshaft includes a torque feature arranged to receive a mating tool which, when engaged with said torque feature and operated, acts to rotate said shaft.

37. The anchor of claim 36, further comprising a counter-rotation feature coupled to said anchor which, when held stationary while said mating tool is operated, prevents said body portions from rotating along with said camshaft.

38. The anchor of claim 35, wherein the shape of each of the camshaft surfaces which force said body portions away from said camshaft have a variable ramp.

39. The anchor of claim 33, wherein said anchor is arranged such that said shaft is inhibited from returning to said first position after said anchor has been activated.

40. The anchor of claim 39, wherein said shaft includes mating flats or detents which inhibit its return to said first position after said anchor has been activated.

41. The anchor of claim 33, wherein said anchor is arranged such that said body portions are locked in their extended positions or inhibited from returning to their de-activated positions after said anchor has been activated.

42. The anchor of claim 33, wherein each of said body portions include a pin and teeth, said anchor arranged such that the pin of one body portion engages the teeth of another body portion to form one or more ratchet arrangements which inhibit said body portions from returning to their de-activated positions after said anchor has been activated.

43. The anchor of claim 33, wherein at least one of said body portions includes an uneven face portion which engages said bone and/or soft tissue and tends to increase said retention force when said anchor is installed within said tissue and in said activated state.

44. The anchor of claim 33, wherein said central shaft is a screw having a diameter that varies along its length, said anchor arranged such that, when de-activated, said screw is in a first position such that said body portions are not extended away from said central shaft, and when activated, said screw is rotated such that said body portions are forced away from said shaft.

45. The anchor of claim 33, wherein said central shaft comprises leaf springs made from a shape-changing material capable of being transformed from a first, pre-formed shape to a second, expanded shape when said anchor is activated, said anchor arranged such that, when de-activated, said leaf springs are in said first, pre-formed shape such that said body portions are not extended away from said central shaft, and when activated, said leaf springs are in said second, expanded shape such that said body portions are forced away from said shaft.

46. The anchor of claim 45, wherein said leaf springs comprise Nitinol and said anchor is activated by increasing the temperature of said leaf springs.

47. A shape-changing anatomical anchor useful for the fixation of bone and soft tissue, said anchor having activated and de-activated states, comprising:
a central drive shaft; and
at least one spike disposed around said drive shaft, each of said spikes mechanically coupled to said drive shaft and arranged to pivot about a pivot point such that, when de-activated, said drive shaft is in a first position such that said at least one spike is not extended away from said central drive shaft, and when activated, said drive shaft is rotated such that said spikes are made to pivot about their pivot points and extend away from said shaft and thereby change the shape of said anchor;
said anchor suitable for installation within said bone and/or soft tissue when in said de-activated state, said shape change when activated tending to increase the force with which said anchor is retained within said tissue.

48. The anchor of claim 47, further comprising:
a top cap; and
at least one planar surface on which at least one of said pivot points is located and at least one of said spikes resides, said planar surfaces being substantially parallel to said top cap and positioned at respective fixed distances below said cap, said central drive shaft passing through said planar surfaces.

49. The anchor of claim 47, wherein said drive shaft includes one or more gears and each of said spikes includes a gear which meshes with a respective one of said drive shaft gears to effect said mechanical coupling.

50. The anchor of claim 47, wherein said drive shaft includes a torque feature arranged to receive a mating tool which, when engaged with said torque feature and operated, acts to rotate said shaft.

51. The anchor of claim 50, further comprising a counter-rotation feature coupled to said anchor which, when held stationary while said mating tool is operated, prevents said spikes from rotating around said shaft when said shaft is rotated.

52. A shape-changing anatomical anchor useful for the fixation of bone and soft tissue, said anchor having activated and de-activated states, comprising:
a central drive shaft; and
at least one spike mechanically coupled to said drive shaft and arranged such that, when de-activated, said drive shaft is in a first position such that said at least one spike is not extended away from said central drive shaft, and when activated, said drive shaft is moved vertically along its longitudinal axis such that said spikes move in a vertical plane parallel to said longitudinal axis to extend away from said shaft and thereby change the shape of said anchor;
said anchor suitable for installation within said bone and/or soft tissue when in said de-activated state, said shape change when activated tending to increase the force with which said anchor is retained within said tissue.
